(19) United States
(12) Patent Application Publication
(10) Pub. No.: US 2021/0186317 A1
(43) Pub. Date: Jun. 24, 2021

(54) INSTRUMENT HANDLE WITH COMPONENT HOLDER

(71) Applicants: JOSHUA MELTON, Pittsburgh, PA (US); RYAN ALLEN, Kaysville, UT (US); BRADLEY P. GRIFFIN, GREENVILLE, NC (US)

(72) Inventors: JOSHUA MELTON, Pittsburgh, PA (US); RYAN ALLEN, Kaysville, UT (US); BRADLEY P. GRIFFIN, GREENVILLE, NC (US)

(73) Assignee: AM Dental LLC, Kaysville, UT (US)

(21) Appl. No.: 17/128,111

(22) Filed: Dec. 20, 2020

Related U.S. Application Data
(60) Provisional application No. 62/950,894, filed on Dec. 19, 2019.

(51) Int. Cl. A61B 1/247; A61C 17/00

(52) U.S. Cl. CPC A61B 1/247; A61C 5/42; A61C 17/005

(57) ABSTRACT

An instrument handle is provided. The instrument handle includes an elongated structure extending between a first end and a second end, the first end extending to a functional head component or is configured to couple to a removable functional head component, and the elongated structure is interrupted by a holding assembly having a through-hole configured to receive a solid object. The holding assembly can have one or more connectors configured to securably retain the solid object therein. A dental mirror including the instrument handle is also provided.

### BACKGROUND

The present disclosure relates to a dental instrument and methods of using the same, and more particularly to a dental mirror handle with additional functionality.

### SUMMARY

The foregoing general summary is intended to provide an overview or framework for understanding the nature and character of the embodiments disclosed herein. This summary is not intended to identify essential inventive concepts of the claimed subject matter or limit the scope of the claimed subject matter. Additional features and advantages of the embodiments disclosed herein will be set forth in the detailed description that follows, and in part will be clear to those skilled in the art from that description or recognized by practicing the embodiments described herein, including the detailed description that follows, the claims, and the appended drawings.

In various embodiments, an instrument handle is provided. The instrument handle includes an elongated structure extending between a first end and a second end, the first end extending to a functional head component or is configured to couple to a removable functional head component, and the elongated structure is interrupted by a holding assembly having a through-hole configured to receive a solid object. The holding assembly can have one or more connectors configured to securably retain the solid object therein.

In various embodiments, a dental mirror is including the instrument handle is also provided. In some embodiments, the holding assembly comprises one or more connectors configured to securably retain the solid object therein.

### BRIEF DESCRIPTION OF THE DRAWINGS

A complete understanding of the present embodiments and the advantages and features thereof will be more readily understood by reference to the following detailed description, appended claims, and accompanying drawings, wherein:

FIG. 1 shows a side view of an instrument handle and dental mirror according to embodiments described herein;

FIG. 2 shows a top view of the instrument handle and dental mirror of FIG. 1;

FIG. 3 shows a partial perspective view of a holding assembly of the instrument handle or FIG. 1;

FIG. 4 shows a top view of a holding assembly on the instrument handle of FIG. 1;

FIG. 5 shows a sectional view of the holding assembly along the A-A line shown in FIGS. 2 and 4;

FIG. 6 shows a sectional view of the holding assembly along the B-B line shown in FIG. 4;

FIG. 7 illustrates a top environmental view of a holding assembly on the instrument handle of FIG. 1;

FIG. 8 illustrates a perspective environmental view of a holding assembly on the instrument handle of FIG. 1;

FIG. 9 shows a side view of an instrument handle and dental mirror according to embodiments described herein;

FIG. 10 shows a top view of the instrument handle and dental mirror of FIG. 9;

FIG. 11 shows a partial perspective view of a holding assembly of the instrument handle or FIG. 9;

FIG. 12 shows a sectional view of the holding assembly along the C-C line shown in FIG. 10;

FIG. 13 a top view of an instrument handle and dental mirror according to embodiments described herein;

FIG. 14 shows a holding assembly and dental mirror according to some embodiments described herein;

FIG. 15A shows a partial top view of an instrument handle and dental mirror, according to some embodiments disclosed herein;

FIG. 15B shows a top view of an alternative holding assembly for the instrument handle and dental mirror in FIG. 15A;

FIG. 15C shows a top view of an alternative holding assembly for the instrument handle and dental mirror in FIG. 15A; and

FIG. 15D shows a side view of the holding assembly in FIG. 15C.

The drawings are not necessarily to scale, and certain features and certain views of the drawings may be shown exaggerated in scale or in schematic in the interest of clarity and conciseness.

### DETAILED DESCRIPTION

Reference will now be made in detail to the exemplary embodiment(s), examples of which is/are illustrated in the accompanying drawings. Whenever possible, the same reference numerals will be used throughout the drawings to refer to the same or like parts.

Before describing the exemplary embodiments, it is noted the embodiments reside primarily in combinations of components and procedures related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The specific details of the various embodiments described herein are used for demonstration purposes only, and no unnecessary limitation or inferences are to be understood therefrom. Furthermore, as used herein, relational terms, such as “first” and “second,” “top” and “bottom,” and the like, may be used solely to distinguish one entity or element from another entity or element without necessarily requiring or implying any physical or logical relationship, or order between such entities or elements.

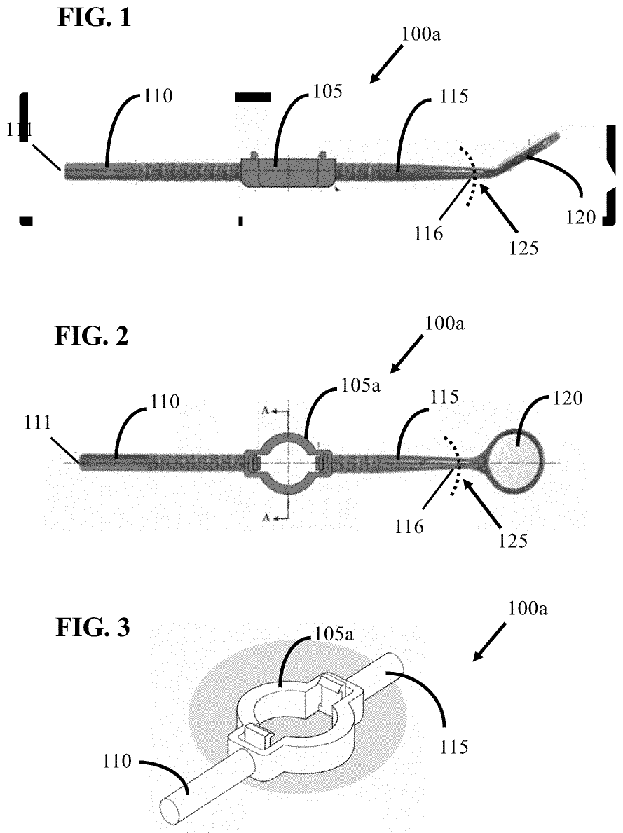

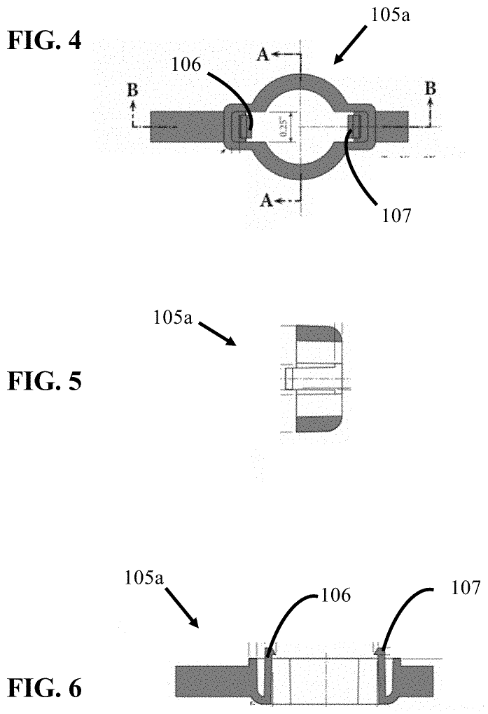

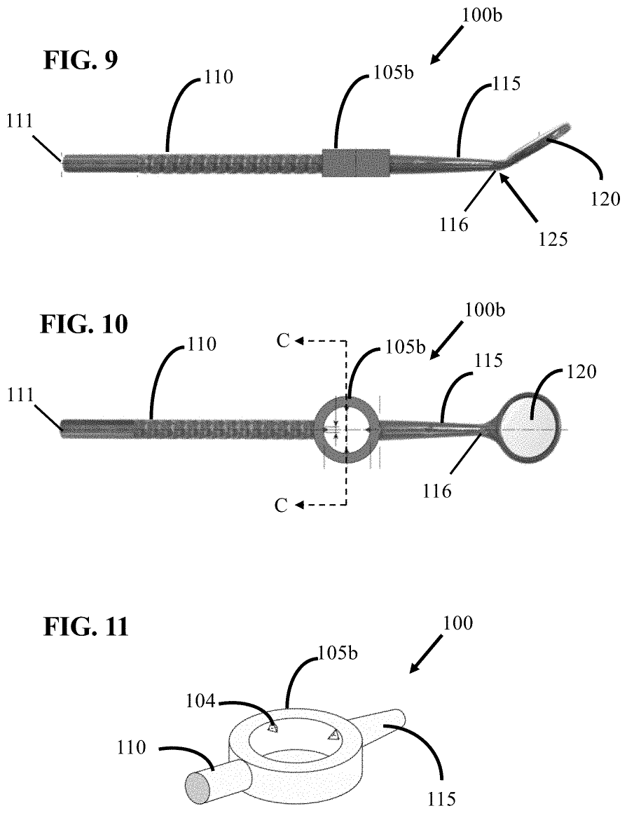

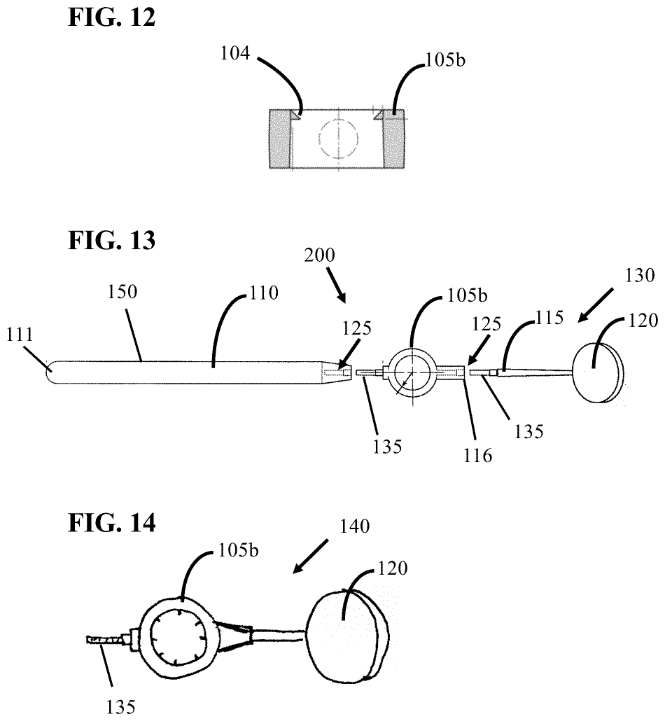

In various embodiments, a multifunctional instrument handle and dental mirror comprising the same is provided. FIGS. 1-15D illustrate the various features included with some embodiments described herein. For example, FIGS. 1, 2, 9, 10, and 13 show an instrument handle 100 in the form of a dental mirror. In some embodiments, the instrument handle 100 (e.g., 100a, 100b, 100c, 100d) comprises an elongated structure extending from a first end 116 to a second end 111. In various embodiments, the elongated structure is interrupted by a holding assembly 105 (e.g., 105a, 105b, 105c, 105d, 105e) having a through-hole configured to receive a solid object. In some embodiments, the elongated structure comprises a first portion 115 extending between the first end 116 and the holding assembly 105, and a second portion 110 extending between the holding assembly 105 and the second end 111.

The instrument handle 100 can be any suitable length. In particular, the first portion 115 and the second portion 110 can be any suitable length, and the holding assembly 105 can be located at any suitable position between the first and second portions 115, 110, as shown in FIGS. 3 and 11. The first portion 115 and the second portion 110 of the elongated instrument handle 100 can have any suitable cross-sectional shape, including, for example, cylindrical, hexagonal, square, triangular, etc. In some embodiments, as shown in FIGS. 1-3, 7-11, 13, and 15A, the first and second portions 115, 110 have a cylindrical or generally cylindrical shape.

In some embodiments, the first and second portions 115, 110 of the instrument handle 100 can have any suitable surface texture. For example, in some embodiments, as shown in FIGS. 3-6, 11, and 13, the first and second portions 115, 110 have a smooth surface. In some embodiments, as shown in FIGS. 1, 2, 9, and 10, the first and second portions 115, 110 can have a rough surface. In such embodiments, the rough surface covers the entire surface of the first and second portions 115, 110. In some embodiments, the rough surface covers only a section of the surface for each of the first and second portions 115, 110. In such embodiments, the rough surface can cover a majority of the entire surface, a minority of the entire surface, less than the entire surface, or at least part of the surface for each of the first and second portions 115, 110. In some embodiments, the rough surface can include any suitable pattern (e.g., lines and rows of dimples, spiral grooves, ribs, etc.). In this context, a suitable pattern is one that would provide the user with a sufficient gripping surface to hold the instrument handle 100 during use.

The holding assembly 105 interrupting the first and second portions 115, 110 of the elongated structure can be any suitable shape and size. In some embodiments, as shown in the overhead and perspective views in FIGS. 2-4, 7, and 8, the holding assembly 105 has a circular or generally circular shape. Such embodiments are particularly useful for holding cylindrically shaped solid objects, including, for example, a cup of toothpaste for an electric toothbrush. Other shapes for the holding assembly 105 are contemplated, including, for example, triangular, square, pentagonal, hexagonal, etc.

In various embodiments, the holding assembly 105 is connected to the first and second portions 115, 110 at opposing sides of the holding assembly. In such embodiments, because the instrument handle 100 has an elongate structure, the first and second portions 115, 110 contact and/or connect to the holding assembly 105 along the longitudinal axis of the elongate structure.

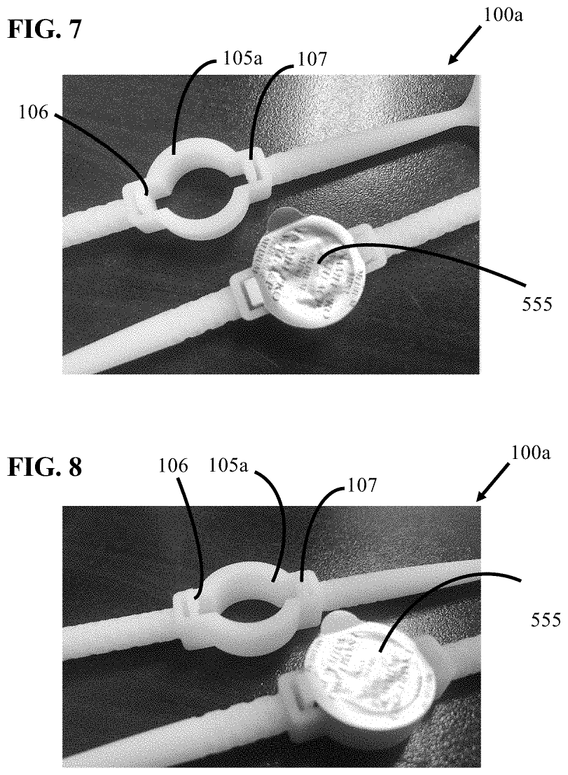

In some embodiments, the holding assembly 105 comprises one or more connectors configured to securably retain a solid object therein. For example, in some embodiments, the holding assembly 105a comprises one or more, or a pair of, connectors configured to securably retain the circular shaped solid object therein. In some embodiments, as shown in FIGS. 1-8, for example, each connector 106, 107 is a snap joint comprising a protruding member that is temporarily deflected to a stressed position while the solid object is inserted into the through-hole, and which returns to a stress-free position after the holding assembly has been joined to the solid object. In some embodiments, each connector 106, 107 is a cantilever snap-fit joint comprising a protruding member that securably retains the solid object after the solid object has been inserted into the through-hole. In some embodiments, the solid object is a circular shaped container. For example, FIGS. 7 and 8 show a container 555 inserted into the holding assembly, and the protruding member of each of the cantilever snap-fit connectors 106, 107 has a lip that reaches over and retains the top surface of the container 555.

In some embodiments, the holding assembly 105a-105e comprises one or more tapered pin(s) 104. In such embodiments, the pins 104 project out from the inner surface of the holding assembly from a one or more spaced apart positions. During use, when a component is inserted into the holding assembly, the component will be releasably held in place by the pins 104. For example, if the component is a container having a conical structure tapering from top to bottom, the component will be insertable into the holding assembly 105 and will slide further into the holding assembly until the tapered surface of the container has a larger diameter than the diameter defined by the pins 104.

In some embodiments, the instrument handle 100a is connected to a functional head component 120. As shown in FIGS. 1 and 2, for example, the functional head component 120 is configured for a dental mirror. In such embodiments, the functional head component 120 comprises a reflective surface. For example, in FIGS. 1 and 2, the first end 116 extends to the functional head component 120 comprising a reflective surface (a mirror component).

In various embodiments, the functional head component 120 is integrated into the first portion 115 during the manufacturing process. In some embodiments, the first and second portions 115, 110, the holding assembly 105, and the functional head component 120 are integrated as a unitary piece during the manufacturing process. For example, the first and second portions 115, 110, the holding assembly 105, and the functional head component 120 are integrated as a unitary piece using a single mold during the manufacturing process.

In some embodiments, the functional head component 120 is manufactured as a separate component. In such embodiments, the functional head component 120 comprises a coupling component configured for coupling the head component 120 to the first portion 115. In such embodiments, the functional head component 120 is a removable functional head component that can be attached and detached from the instrument handle 100 at the first end 116. For example, FIGS. 13-15A each show an independent functional head component 130 having a connecting component 135 that can be joined with a complementary connecting component 125 located at the first end 116, as shown in FIGS. 1, 2 and 13. In some embodiments, the connecting component 135 is a threaded post, which is configured to be threaded into a complementary threaded hole 125 located at the first end 116 of the instrument handle 100. In such embodiments, the threaded component 135 and the instrument handle 100 are removably joined to form a unitary instrument.

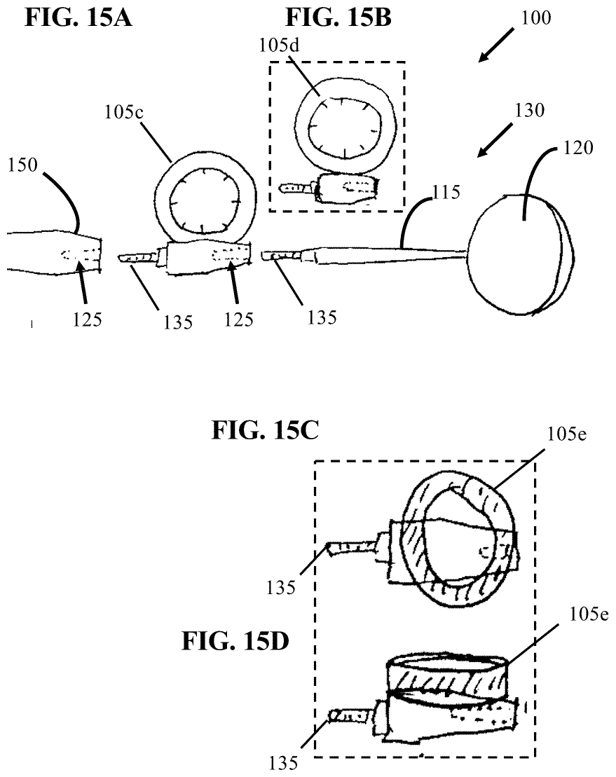

In some embodiments, as shown in FIGS. 13 and 15A-15E, a dental mirror 200 is provided. In such embodiments, the holding assembly 105 is a separate component from a shank component 150 and the functional head component 120. The holding assembly 105 can be configured with various features. For example, as shown in FIG. 13, the holding assembly 105b comprises a threaded hole 125 on one side and a threaded post 135 on the opposite side. In such embodiments, the threaded hole 125 is configured to receive a threaded post 135 from the functional head component 130, and the shank 150 comprises a threaded hole 125 configured to receive the threaded post 135 on the holding assembly 105b. As shown in FIG. 13, the circular shape of the holding assembly 105b intercepts the elongated structure of the dental instrument.

In some embodiments, as shown in FIG. 14, the functional head component 120 is combined with the holding assembly 105 to form a multifunctional component 140. In such embodiments, the multifunctional component 140 comprises a threaded post 135 for connecting to a shank 150.

In some embodiments, as shown in FIGS. 15A-15D, the holding assembly 105 can have any suitable orientation when configured to be joined on one end with a shank 150 and on the opposing end with a functional head component 120. For example, the holding assembly 105c in FIG. 15A comprises an opening for receiving a component that is merged into a body component; the holding assembly 105d is joined to a side of the body component, and in FIGS. 15C and 15D, the holding assembly 105e is joined a surface of the body component.

In some embodiments, the functional head component 120 comprises the first portion 115, which projects away from the first end 116 along a longitudinal axis defined by the elongated structure before projecting at an angle between 10 and 70 degrees relative to the longitudinal axis defined by the elongated structure. For example, the side view in FIG. 1 shows the functional head component at an angle ranging from about 20 degrees to 50 degrees.

In some embodiments, the instrument handle 100 is integrated into a dental instrument. For example, a dental mirror comprising the instrument handle 100 is contemplated. In such embodiments, the dental mirror comprises a functional head component 120 having a reflective surface and the instrument handle 100. In such embodiments, the instrument handle 100 comprises an elongated structure extending between a first end 116 and a second end 111, wherein the first end 116 extends to the functional head component 120. In such embodiments, the elongated structure of the instrument handle 100 is interrupted by a holding assembly 105 having a through-hole configured to retain a solid object.

In some embodiments, the holding assembly 105 comprises one or more connectors configured to securably retain the solid object therein. In some embodiments, each connector is a snap joint comprising a protruding member that is temporarily deflected to a stressed position while the solid object is inserted into the through-hole, and which returns to a stress-free position after the holding assembly has been joined to the solid object. For example, in FIGS. 1-8, the holding assembly 105 comprises a pair of connectors 106, 107, and each connector is a cantilever snap-fit comprising a protruding member that securably retains the solid object after the solid object has been inserted into the through-hole.

In some embodiments, the dental instrument comprises a functional head component 120 that is sized to fit inside a human mouth. In some embodiments, for example, the functional head component 120 is a universal, small, medium, or large size.

In some embodiments, the instrument handle 100 comprises a measuring component. For example, in some embodiments, a surface of the instrument handle 100 comprises a ruler. In some embodiments, the ruler is sufficiently long enough to measure a root canal file, or the working portion of a root canal file. During a root canal procedure, the user may hold the instrument handle 100 in one hand and the root canal instrument in the other canal. Occasionally, as the user works through the canals of a tooth, there is a need to measure how far the root canal file has penetrated into the tooth. Accordingly, having a ruler on the instrument handle 100 is very convenient for measuring instruments such as a root canal file.

The instrument handle disclosed herein can be comprised of any suitable material. Those skilled in the art would appreciate the material suitable for medical use. Such materials include surgical grade stainless steel, plastics, etc. In some embodiments, the instrument handle is designed and manufactured as a single-use instrument, as a multi-use instrument that can be sterilized before use (e.g., autoclave, ethylene oxide), or a long-term use instrument that can be sterilized before use.

The instrument handle disclosed herein, having a functional head with a reflective surface or otherwise, provides a convenient holding assembly for items needed during a procedure, such as a dental procedure. For example, when a dental hygienist is cleaning a patient's teeth with an electric toothbrush, both of their hands are usually occupied, with one hand holding the toothbrush and the other hand holding a dental (mouth) mirror. When the toothbrush needs to be refreshed with additional toothpaste, the hygienist must pivot away from the patient to replenish the toothpaste. The instrument handle provided herein, however, allows the hygienist to replenish the toothpaste without moving away from the patient's mouth. During use, their hand holding the toothbrush will be able to easily access additional toothpaste from the holding assembly located on the instrument handle, which is held in their other hand.

Other uses of the holding assembly located on the instrument handle are contemplated. For example, the holding assembly is useful for retaining a sponge adapted for cleaning the tip of an instrument. During a procedure, the user will be able to continue the procedure without disruption when the instrument needs to be cleaned. The holding assembly on the instrument handle is further useful for holding restoration materials, such as cements and liquids, or endodontic files and/or measuring instruments used during a root canal procedure.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

1. An instrument handle comprising an elongated structure extending between a first end and a second end;
wherein the first end extends to a functional head component or is configured to couple to a removable functional head component, and
wherein the elongated structure is interrupted by a holding assembly having a through-hole configured to receive a solid object.

2. The instrument handle of claim 1, wherein the holding assembly comprises one or more connectors configured to securably retain the solid object therein.

3. The instrument handle of claim 1, wherein the holding assembly comprises a circular shape configured to receive a circular shaped solid object.

4. The instrument handle of claim 3, wherein the holding assembly comprises a pair of connectors configured to securably retain the circular shaped solid object therein.

5. The instrument handle of claim 4, wherein the circular shaped solid object is a container comprising toothpaste.

6. The instrument handle of claim 1, wherein the elongated structure comprises a first portion extending between the first end and the holding assembly, and a second portion extending between the holding assembly and the second end.

7. The instrument handle of claim 6, wherein each of the first and second portions of the elongate structure comprises a cylindrical cross-sectional shape.

8. The instrument handle of claim 2, wherein each connectors is a snap joint comprising a protruding member that is temporarily deflected to a stressed position while the solid object is inserted into the through-hole, and which returns to a stress-free position after the holding assembly has been joined to the solid object.

9. The instrument handle of claim 2, wherein each connector is a cantilever snap-fit comprising a protruding member that securably retains the solid object after the solid object has been inserted into the through-hole.

10. The instrument handle of claim 1, wherein the elongated structure comprises a first portion extending between the first end and the holding assembly, and a second portion extending between the holding assembly and the second end;
wherein the holding assembly comprises a pair of connectors configured to securably retain the solid object therein; and
wherein the pair of connectors are positioned on opposing sides of the holding assembly at positions corresponding to where the first and second portions of the elongated structure contact the holding assembly.

11. The instrument handle of claim 1, wherein the functional head component or the removable functional head component comprises a mirrored surface.

12. The instrument handle of claim 11, wherein the functional head component or the removable functional head component projects away from the first end along a longitudinal axis defined by the elongated structure before projecting at an angle between 10 and 70 degrees relative to the longitudinal axis defined by the elongated structure.

13. The instrument handle of claim 1, wherein the first end comprises a threaded component configured to couple with the removable functional head component having a complementary threaded component, wherein the threaded component and the complementary threaded component are joinable to form a unitary instrument.

14. (canceled)

15. The instrument handle of claim 1, wherein the elongated structure, the holding assembly, and the removable functional head component are manufactured as separate components using separate molds and then joined to form the dental mirror.

16. A dental mirror comprising an instrument handle and a functional head component having a reflective surface;
wherein the instrument handle comprises an elongated structure extending between a first end and a second end;
wherein the first end extends to the functional head component; and
wherein the elongated structure is interrupted by a holding assembly having a through-hole configured to retain a solid object.

17. The dental mirror of claim 16, wherein the holding assembly comprises one or more connectors configured to securably retain the solid object therein.

18. The dental mirror of claim 17, wherein each connector is a snap joint comprising a protruding member that is temporarily deflected to a stressed position while the solid object is inserted into the through-hole, and which returns to a stress-free position after the holding assembly has been joined to the solid object.

19. The dental mirror of claim 17, wherein each connector is a cantilever snap-fit comprising a protruding member that securably retains the solid object after the solid object has been inserted into the through-hole.

20. The dental mirror of claim 16, wherein the functional head component is a removable functional head component.

21. The dental mirror of claim 16, wherein the functional head component comprises a circular shape and is sized to fit inside a human mouth.

22. (canceled)
